(19) United States
(12) Patent Application Publication
(10) Pub. No.: US 2025/0255554 A1
(43) Pub. Date: Aug. 14, 2025

(54) DETERMINATION OF CARDIAC FLOW RATE WITH AN IMPLANTABLE MEDICAL DEVICE

(71) Applicant: Medtronic, Inc., Minneapolis, MN (US)

(72) Inventor: Nirav A. Patel, Dublin, OH (US)

(21) Appl. No.: 18/857,660

(22) PCT Filed: Apr. 4, 2023
(86) PCT No.: PCT/IB2023/053416

§ 371 Date: Oct. 17, 2024

Related U.S. Application Data
(60) Provisional application No. 63/363,459, filed on Apr. 22, 2022.

(51) Int. Cl. A61B 5/00; A61B 5/0205; A61B 5/026; A61B 5/07; A61B 5/11; A61B 5/145; A61B 5/1455; A61B 5/1459

(52) U.S. Cl. CPC A61B 5/6861; A61B 5/0205; A61B 5/0261; A61B 5/076; A61B 5/7267; A61B 5/7278; A61B 5/7282; A61B 5/746; A61B 5/747; A61B 5/1116; A61B 5/1118; A61B 5/14535; A61B 5/14552; A61B 5/1459; A61B 2562/16

(57) ABSTRACT

This disclosure is directed to devices, systems, and techniques for determining cardiac flow rate. An example medical device is configured to be at least substantially entirely inserted into a vasculature of a patient. The medical device includes an elongated housing and a first optical sensor configured to sense a first red blood cell count and generate a first signal indicative of the first red blood cell count. The medical device includes a second optical sensor configured to sense a second red blood cell count and generate a second signal indicative of the second red blood cell count, the second optical sensor being longitudinally displaced from the first optical sensor. The medical device also includes processing circuitry configured to determine a cardiac flow rate based on a first signal and the second signal.

This application claims the benefit of U.S. Provisional Application Ser. No. 63/363,459, filed Apr. 22, 2022, the entire content of which is incorporated herein by reference.

### TECHNICAL FIELD

The disclosure relates generally to medical devices and, more particularly, medical devices configured to monitor patient parameters.

### BACKGROUND

Some types of medical devices may be used to monitor one or more physiological parameters of a patient. Such medical devices may include, or may be part of a system that includes, sensors that detect signals associated with such physiological parameters. Values determined based on such signals may be used to assist in detecting changes in patient conditions, in evaluating the efficacy of a therapy, or in generally evaluating patient health.

### SUMMARY

In general, the disclosure is directed to devices, systems, and techniques for using an implantable medical device to determine a cardiac flow rate of a patient. Cardiac flow rate, especially during patient activity, may be an important indicator of relative patient wellbeing for heart failure patients, peripheral vascular disease patients, diabetes patients (who may have deteriorating vascular disease), patients at risk of deep vein thrombosis, and other cardiac patients. For example, worsening heart failure may be detected by a slowing of the cardiac flow rate or cardiac flow rate may acutely drop in a patient with deep vein thrombosis. The techniques of this disclosure allow for remote, ambulatory, continuous or chronic measurement of cardiac flow rate.

Cardiac flow rate may be used by a clinician to guide therapy and/or medication for such patients. Therefore, a continuous (e.g., on a periodic or triggered basis without human intervention), chronic measurement of cardiac flow rate may be important to patient health. Currently, however, cardiac flow rate is typically measured in a laboratory, hospital, or other clinical setting by inserting a catheter into the vasculature of the patient to measure cardiac flow rate acutely. Such techniques do not typically provide for a remote, ambulatory, continuous, or chronic measurement of cardiac flow.

According to the techniques of this disclosure, a medical device may be an insertable medical device that may be at least substantially entirely (e.g., wholly) inserted into the vasculature of a patient to continuously monitor cardiac flow rate in the patient. The medical device may include an elongated housing. The medical device may include a first optical sensor and a second optical sensor. The first and second optical sensors may each be configured to sense a red blood cell count in blood flowing proximate the optical sensor. The first optical sensor may be disposed on the medical device on a first portion of the medical device. The second optical sensor may be disposed on a second portion of the medical device that is longitudinally displaced from the first portion of the medical device, such that the first optical sensor and the second optical sensor are separated by a distance. For example, the first optical sensor may be located a fixed distance from the second optical sensor along a length of the housing of the medical device. Each of the first optical sensor and the second optical sensor may generate a signal indicative of the red blood cell count. Processing circuitry may compare the red blood cell counts and determine a cardiac flow rate based on the signals from the first optical sensor and the second optical sensor. Such a medical device and techniques may facilitate the remote, ambulatory, continuous, or chronic monitoring of cardiac flow in the patient. While generally discussed herein as including a first optical sensor and a second optical sensor which may generate a signal indicative of red blood cell counts, the techniques of this disclosure may include the use of sensors more generally which may be configured to sense vasculature particle counts, such as red blood cell counts, white blood cell counts, and/or platelet counts.

In some examples, the techniques and systems of this disclosure may use a machine learning model to more accurately determine patient health status based on physiological data collected by an IMD. In some examples, the machine learning model is trained with a set of training instances, where one or more of the training instances comprise data that indicate relationships between various cardiac flow rates and health states (e.g., health event likelihoods). Because the machine learning model is trained with potentially thousands or millions of training instances, the machine learning model may reduce the amount of error in determining health states. Reducing errors using the techniques of this disclosure may provide one or more technical and clinical advantages, such as increasing reliability of another device, user, and/or clinician on the accuracy of determining a patient's condition and improve resulting treatment of the patient and patient outcomes.

In some examples, a medical device configured to be at least substantially entirely inserted into a vasculature of a patient includes: an elongated housing; a first optical sensor configured to sense a first red blood cell count and generate a first signal indicative of the first red blood cell count, the first optical sensor being disposed on a first portion of the medical device; a second optical sensor configured to sense a second red blood cell count and generate a second signal indicative of the second red blood cell count, the second optical sensor being disposed on a second portion of the device, the second portion of the device being longitudinally displaced from the first portion of the device; and processing circuitry configured to determine a cardiac flow rate based on a first signal and the second signal.

In some examples, a method includes sensing, by a first optical sensor, a first red blood cell count; generating, by the first optical sensor, a first signal indicative of the first red blood cell count, the first optical sensor being disposed on a first portion of a medical device having an elongated housing and being configured to be at least substantially entirely inserted within a vasculature of a patient; sensing, by a second optical sensor, a second red blood cell count; generating, by the second optical sensor, a second signal indicative of the second red blood cell count, the second optical sensor being disposed on a second portion of medical device, the second portion of the device being longitudinally displaced from the first portion of the medical device; and determining, by processing circuitry, a cardiac flow rate based on a first signal and the second signal.

In some examples, a non-transitory computer-readable medium includes instructions for causing one or more processors to: determine a cardiac flow rate based on a first signal and a second signal, wherein the first signal is from a first optical sensor disposed on a first portion of a medical device having an elongated housing and being configured to be at least substantially entirely inserted within a vasculature of a patient, the first signal being indicative of a first red blood cell count, and wherein the second signal is from a second optical sensor disposed on a second portion of medical device, the second signal being indicative of a second red blood cell count, the second portion of the device being longitudinally displaced from the first portion of the medical device.

In some examples, a medical device is configured to be at least substantially entirely inserted into a vasculature of a patient, the medical device including an elongated housing; a first sensor configured to sense a first vascular particle count and generate a first signal indicative of the first vascular particle count, the first sensor being disposed on a first portion of the medical device; a second sensor configured to sense a second vascular particle count and generate a second signal indicative of the second vascular particle count, the second sensor being disposed on a second portion of the medical device, the second portion of the medical device being longitudinally displaced from the first portion of the medical device; and processing circuitry configured to determine a cardiac flow rate based on a first signal and the second signal.

This summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 illustrates the environment of an example medical device system in conjunction with a patient, in accordance with one or more techniques of this disclosure.

FIG. 2 is a conceptual drawing illustrating an example configuration of the implantable medical device (IMD) of the medical device system of FIG. 1, in accordance with one or more techniques described herein.

FIG. 3 is a functional block diagram illustrating an example configuration of the IMD of FIGS. 1 and 2, in accordance with one or more techniques described herein.

FIGS. 4A and 4B are block diagrams illustrating two additional example IMDs that may be substantially similar to the IMD of FIGS. 1-3, but which may include one or more additional features, in accordance with one or more techniques described herein.

FIG. 5 is a block diagram illustrating an example configuration of components of the external device of FIG. 1, in accordance with one or more techniques of this disclosure.

FIG. 6 is a block diagram illustrating an example system that includes an access point, a network, external computing devices, such as a server, and one or more other computing devices, which may be coupled to the IMD of FIGS. 1-4B, an external device, and processing circuitry via a network, in accordance with one or more techniques described herein.

FIG. 7 is a flow diagram illustrating an example of cardiac flow rate determination techniques according to one or more aspects of this disclosure.

FIG. 8 is a conceptual diagram illustrating an example machine learning model configured to determine an extent to which data of a patient indicates an acute health event.

FIG. 9 is a conceptual diagram illustrating an example training process for an artificial intelligence model, in accordance with examples of the current disclosure.

Like reference characters denote like elements throughout the description and figures.

### DETAILED DESCRIPTION

This disclosure describes techniques for determining a cardiac flow rate of a patient. Cardiac flow rate is a physiological parameter that clinicians, such as cardiologists, may use to manage or titrate heart failure medication, such as angiotensin-converting enzyme (ACE)-inhibitors and/or angiotensin II receptor blockers (ARBs) for hyperkalemia, diuretics for hypokalemia, beta-blockers, or the like, and/or therapies. The techniques of this disclosure may facilitate the remote, ambulatory, continuous, or chronic monitoring and management of a heart failure patient, peripheral vascular disease patient, diabetes patient, patient at risk of deep vein thrombosis, and/or other cardiac patient. By providing for the remote, ambulatory, continuous, or chronic monitoring of cardiac flow rate, the techniques of this disclosure may facilitate early intervention by a clinician during deteriorating conditions of the patient, faster detection of actionable events, reduced hospitalization, better management of medications, such as heart failure medications which may improve patient outcomes and patient quality of life.

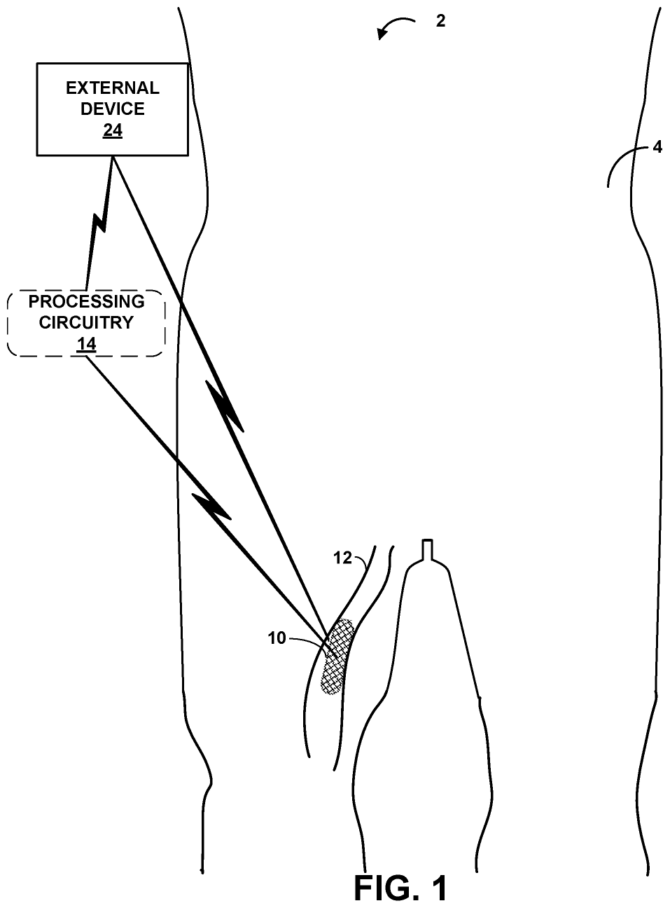

FIG. 1 illustrates the environment of an example medical device system 2 in conjunction with a patient 4, in accordance with one or more techniques of this disclosure. While the techniques described herein are generally described in the context of an insertable medical device, such as an insertable cardiac monitor, the techniques of this disclosure may be implemented in any implantable medical device (IMD) configured to be introduced into the vasculature of a patient and configured to sense a red blood cell count of the patient at two different locations on the medical device. The example techniques may be used with an IMD 10, which may be in wireless communication with at least one of external device 24 and other devices not pictured in FIG. 1. Processing circuitry 14 is conceptually illustrated in FIG. 1 as separate from IMD 10 and external device 24, but may be processing circuitry of IMD 10 and/or processing circuitry of external device 24. In general, the techniques of this disclosure may be performed by processing circuitry 14 of one or more devices of a system, such as one or more devices that include sensors that provide signals, or processing circuitry of one or more devices that do not include sensors, but nevertheless analyze signals using the techniques described herein. For example, another external device (not pictured in FIG. 1) may include at least a portion of processing circuitry 14, the other external device configured for remote communication with IMD 10 and/or external device 24 via a network.

In some examples, IMD 10 is inserted, for example using an insertion catheter or tool, into the vasculature 12 of patient 4. IMD 10 may be elongated and relatively flat so as to permit the flow of blood around a housing of IMD 10. IMD 10 may be inserted into the great saphenous vein, common femoral vein, or another relatively broad vein or artery. In some examples, IMD 10 may be inserted into a relatively broad vein or artery in an arm or leg of patient 4. For a patient with pulmonary embolism in the lungs, IMD 10 may be inserted into the vasculature closer to the heart of the patient, rather than in the arm or leg. Vasculature 12 and IMD 10 are shown enlarged for easier visibility.

Clinicians sometimes diagnose patients with medical conditions based on one or more observed physiological signals collected by physiological sensors, such as optical sensors, electrodes, pressure sensors, chemical sensors, temperature sensors, acoustic sensors, and motion sensors (e.g., accelerometers). In some cases, clinicians apply non-invasive sensors or invasive sensors, such as a cardiac flow rate catheter, to patients in order to sense one or more physiological signals while a patient is in a clinic for a medical appointment. However, in some examples, physiological markers (e.g., arrythmia, etc.) of a patient condition are rare or are difficult to observe over a relatively short period of time. As such, in these examples, a clinician may be unable to observe the physiological markers needed to diagnose comorbidities of a patient with a medical condition or effectively treat the patient while monitoring one or more physiological signals, such as cardiac flow rate, of the patient during a medical appointment. In the example illustrated in FIG. 1, IMD 10 is implanted within patient 4 to record one or more physiological signals, such as a cardiac flow rate, of patient 4 continuously and over an extended period of time.

In some examples, IMD 10 includes a first optical sensor and a second optical sensor, each of which may be configured to sense a red blood cell count in blood passing proximate (e.g., over) the optical sensors. For example, IMD 10 may include one of more light generators that may generate light that is directed away from IMD 10 into vasculature 12 of patient 4. The first optical sensor and the second optical sensor may sense light reflected off of the red blood cells, other components of blood, and/or the surrounding vasculature to determine a red blood cell count. In some examples, IMD 10 may include other sensors configured to sense physiological parameters of patient 4 or that enable processing circuitry of IMD 10 to determine physiological parameters of patient 4. For example, IMD 10 may include one or more accelerometers, pressure sensors, temperature sensors, chemical sensors, and/or a plurality of electrodes. Such sensors may detect one or more physiological parameters indicative of a patient condition.

According to the techniques of this disclosure, IMD 10, external device 24, and/or processing circuitry 14 may use red blood cell count signals generated by the first optical sensor and the second optical sensor to determine a cardiac flow rate of patient 4. For example, IMD 10, external device 24, and/or processing circuitry 14 may compare the red blood cell count signals or the red blood cell counts. When the red blood cell count sensed by the first optical sensor and the red blood cell count sensed by the second optical sensor are equal, that may be indicative that the red blood cells that were located proximate the first optical sensor are now proximate the second optical sensor. Because sometimes red blood cells may collide, IMD 10 may include a buffer amount, which may be a percentage, when determining that the red blood cells that were located proximate the first optical sensor have now reached the second optical sensor. For example, IMD 10 may determine that the red blood cells that were located proximate the first optical sensor are now proximate the second optical sensor when the count of red blood cells at the second optical sensor is within percentage of the count of the red blood cells at the first optical sensor at a previous time. For example, the percentage may be programmable within a range of 5-25%. In some examples, the percentage may be initially set to 10%. IMD 10, external device 24, and/or processing circuitry 14 may determine a length of time that it took for the red blood cells to travel from the first optical sensor to the second optical sensor. As the first optical sensor and the second optical sensor are separated from each other along the length of IMD 10, by a fixed distance, IMD 10, external device 24, and/or processing circuitry 14 may use the fixed distance and the determined length of time to determine a speed at which the red blood cells were traveling. For example, when the sensed value of red blood cell count from the first optical sensor at time X is equal to (or is within the predetermined buffer amount of being equal to) the red blood cell count from a second sensor at time X+Y, the ICM may determine that the red blood cells flowed from the first optical sensor to the second optical sensor in time Y. In some examples, IMD 10, external device 24, and/or processing circuitry 14 may use a formula of flow rates (absolute or relative) at a given pressurized tube state and the speed at which the red blood cells were traveling to calculate or determine the cardiac flow rate.

In some examples, IMD 10 may determine the cardiac flow rate and transmit the determined cardiac flow rate to external device 24 and/or processing circuitry 14. In some examples, IMD 10, external device 24, and/or processing circuitry 14 may compare the determined cardiac flow rate to a predetermined threshold and if the determined cardiac flow rate meets the predetermined threshold, generate an indication for output. In some examples, the indication may be an alert, instructions to seek medical attention, instructions on treatment or a remedial action (e.g., take a dosage of medication, rest, etc.), or information including other sensed physiological parameters (either from other sensors of IMD 10 or from sensor external to IMD 10, such as sensors of other implantable devices or wearable devices).

In some examples, IMD 10, external device 24, and/or processing circuitry 14 may apply at least one machine learning model, e.g., at least one patient specific machine learning model, to determine when it may be appropriate or desirable to generate the indication for output. For example, a patient specific machine learning model may be trained using cardiac flow rate data collected from patient 4 and/or other sensed physiological parameters collected from patient 4. As each patient's cardiac health may be different, the machine learning model may be applicable only to the patient whose data was used to train the model.

In some examples, cardiac flow rates determined for different postures or activities may be used with the machine learning model to determine when it may be appropriate or desirable to generate the indication for output. For example, IMD 10, external device 24, and/or processing circuitry 14 may use one or more motion sensor(s) to determine a posture or activity of patient 4 and may determine a cardiac flow rate for each posture or activity of patient 4, such as a first cardiac flow rate of patient 4 in a supine posture, a second cardiac flow rate of patient 4 in an upright posture, a third cardiac flow rate of patient 4 at rest, a fourth cardiac flow rate of patient 4 in a walking activity, etc. In some examples, IMD 10, external device 24, and/or processing circuitry 14 may determine a periodic (e.g., daily) average of respective cardiac flow rates associated with each posture and/or activity. Such average cardiac flow rates may be used to generate (e.g., by external device 24) a report for a clinician.

Potential machine learning models may be configured as Bayesian algorithms, clustering algorithms, decision-tree algorithms, regularization algorithms, regression algorithms (e.g., linear or logistic), instance-based algorithms, artificial neural network algorithms, deep learning algorithms, random forest algorithms, support vector machines, dimensionality reduction algorithms and the like. Various examples of specific algorithms include Naïve Bayes, Bayesian Linear Regression, Boosted Decision Tree Regression, and Neural Network Regression, Back Propagation Neural Networks, Convolution Neural Networks (CNN), Long Short Term Networks (LSTM), the Apriori algorithm, K-Means Clustering, k-Nearest Neighbour (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

For example, a k-means clustering model may be used having two clusters: one for generating an indication for output and one for not generating an indication for output. Each sensed cardiac flow rate may be associated with a vector that includes variables for, e.g., previous sensed cardiac flow rates, disease state, comorbidities, cholesterol level, age, physical exercise level, etc. The location of the vector in a given one of the clusters may be indicative of whether IMD 10, external device 24, and/or processing circuitry 14 should generate the indication for output. For example, if the vector falls within generate the indication for output cluster, IMD 10, external device 24, and/or processing circuitry 14 may generate the indication for output.

External device 24 may be a hand-held computing device with a display viewable by the user and an interface for providing input to external device 24 (i.e., a user input mechanism). For example, external device 24 may include a small display screen (e.g., a liquid crystal display (LCD) or a light emitting diode (LED) display) that presents information to the user. In addition, external device 24 may include a touch screen display, keypad, buttons, a peripheral pointing device, voice activation, or another input mechanism that allows the user to navigate through the user interface of external device 24 and provide input. If external device 24 includes buttons and a keypad, the buttons may be dedicated to performing a certain function, e.g., a power button, the buttons and the keypad may be soft keys that change in function depending upon the section of the user interface currently viewed by the user, or any combination thereof.

In other examples, external device 24 may be a larger workstation or a separate application within another multi-function device, rather than a dedicated computing device. For example, the multi-function device may be a notebook computer, tablet computer, workstation, one or more servers, cellular phone, personal digital assistant, or another computing device that may run an application that enables the computing device to operate as a secure device.

When external device 24 is configured for use by the clinician, external device 24 may be used to transmit instructions to IMD 10 and to receive measurements, such as a cardiac flow rate of patient 4, or other sensed physiological parameters. Example instructions may include requests to set electrode combinations for sensing and any other information that may be useful for programming into IMD 10. The clinician may also configure and store operational parameters for IMD 10 within IMD 10 with the aid of external device 24. In some examples, external device 24 assists the clinician in the configuration of IMD 10 by providing a system for identifying potentially beneficial operational parameter values.

Whether external device 24 is configured for clinician, patient or caregiver use, external device 24 is configured to communicate with IMD 10 and, optionally, another computing device (not illustrated in FIG. 1), via wireless communication. External device 24, for example, may communicate via near-field communication technologies (e.g., inductive coupling, NFC or other communication technologies operable at ranges less than 10-20 cm) and far-field communication technologies (e.g., RF telemetry according to the 802.11 or Bluetooth® specification sets, or other communication technologies operable at ranges greater than near-field communication technologies).

Processing circuitry 14, in some examples, may include one or more processors that are configured to implement functionality and/or process instructions for execution within IMD 10. For example, processing circuitry 14 may be capable of processing instructions stored in a storage device. Processing circuitry 14 may include, for example, microprocessors, digital signal processors (DSPs), application specific integrated circuits (ASICs), field-programmable gate arrays (FPGAs), or equivalent discrete or integrated logic circuitry, or a combination of any of the foregoing devices or circuitry. Accordingly, processing circuitry 14 may include any suitable structure, whether in hardware, software, firmware, or any combination thereof, to perform the functions ascribed herein to processing circuitry 14.

Processing circuitry 14 may represent processing circuitry located within any combination of IMD 10 and external device 24. In some examples, processing circuitry 14 may be entirely located within a housing of IMD 10. In other examples, processing circuitry 14 may be entirely located within a housing of external device 24. In other examples, processing circuitry 14 may be located within any combination of IMD 10, external device 24, and another device or group of devices that are not illustrated in FIG. 1. As such, techniques and capabilities attributed herein to processing circuitry 14 may be attributed to any combination of IMD 10, external device 24, and other devices that are not illustrated in FIG. 1.

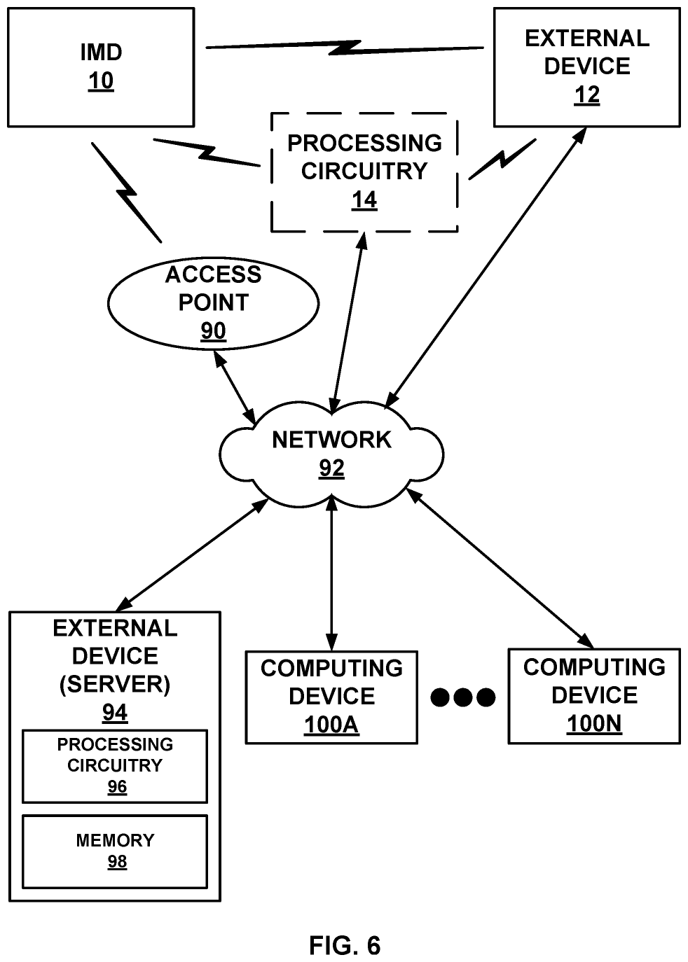

In some examples, IMD 10 includes one or more motion sensors, such as accelerometers. An accelerometer of IMD 10 may collect an accelerometer signal which reflects a measurement of a motion (e.g., activity) or posture of patient 4. In some cases, the accelerometer may collect a three-axis accelerometer signal indicative of patient 4's movements within a three-dimensional Cartesian space. For example, the accelerometer signal may include a vertical axis accelerometer signal vector, a lateral axis accelerometer signal vector, and a frontal axis accelerometer signal vector. The vertical axis accelerometer signal vector may represent an acceleration of patient 4 along a vertical axis, the lateral axis accelerometer signal vector may represent an acceleration of patient 4 along a lateral axis, and the frontal axis accelerometer signal vector may represent an acceleration of patient 4 along a frontal axis. In some cases, the vertical axis substantially extends along a torso of patient 4 when patient 4 from a neck of patient 4 to a waist of patient 4, the lateral axis extends across a chest of patient 4 perpendicular to the vertical axis, and the frontal axis extends outward from and through the chest of patient 4, the frontal axis being perpendicular to the vertical axis and the lateral axis. In some examples, processing circuitry 14 may be configured to identify, based on one of more accelerometer signals, an activity or posture of patient 4. In some examples, processing circuitry 14 may make a plurality of determinations of the cardiac flow rate of the patient in each of a plurality of activities or postures. For example, when patient 4 is in a prone posture, processing circuitry 14 may make a plurality of cardiac flow measurements or determinations. When patient 4 is in an upright posture, processing circuitry 14 may make a plurality of cardiac flow measurements or determinations. Processing circuitry 14 may do the same for other postures or activities. In some examples, processing circuitry 14 may average (e.g., determine a mean, median, mode, or other measure of central tendency) the cardiac flow measurements for each posture on a periodic basis, such as a daily basis, and transmit the average for each posture to an external device, e.g., external device 24 or server 94 (FIG. 6), for generating a report for a clinician.

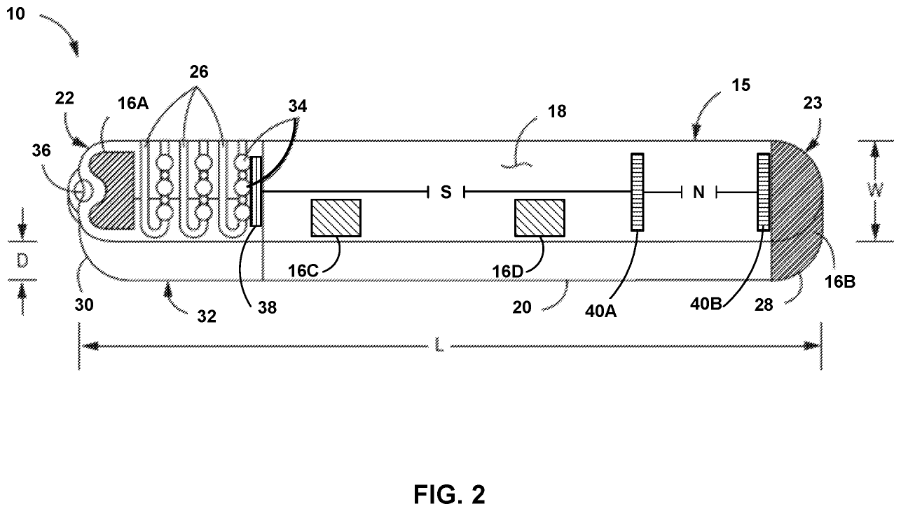

FIG. 2 is a conceptual drawing illustrating an example configuration of IMD 10 of the medical device system 2 of FIG. 1, in accordance with one or more techniques described herein. In the example shown in FIG. 2, IMD 10 may be a leadless, vascularly-implantable monitoring device having housing 15, proximal electrode 16A, and distal electrode 16B. While shown in FIG. 2 as having electrodes, in some examples, the IMD may not include electrodes. Housing 15 may further include first major surface 18, second major surface 20, proximal end 22, and distal end 23. In some examples, IMD 10 may include one or more additional electrodes 16C, 16D positioned on one or both of major surfaces 18, 20 of IMD 10. Housing 15 encloses electronic circuitry located inside the IMD 10, and protects the circuitry contained therein from fluids such as body fluids (e.g., blood). In some examples, electrical feedthroughs provide electrical connection of electrodes 16A-16D, and antenna 26, to circuitry within housing 15. In some examples, electrode 16B may be formed from an uninsulated portion of conductive housing 15.

In the example shown in FIG. 2, IMD 10 is defined by a length L, a width W, and thickness or depth D. In this example, IMD 10 is in the form of an elongated rectangular prism in which length L is significantly greater than width W, and in which width W is greater than depth D. However, other configurations of IMD 10 are contemplated, such as those in which the relative proportions of length L, width W, and depth D vary from those described and shown in FIG. 2. In some examples, the geometry of the IMD 10, such as the width W being greater than the depth D, may be selected to allow IMD 10 to be inserted into vasculature 12 of the patient using a minimally invasive procedure and to remain in a desired orientation during insertion. In some examples, depth D is relatively flat so as to permit blood to flow past (e.g., over) IMD 10 when inserted into vasculature 12 of patient 4. In addition, IMD 10 may include radial asymmetries (e.g., the rectangular shape) along a longitudinal axis of IMD 10, which may help maintain the device in a desired orientation following implantation. In some examples, the shape and size of IMD 10 may be chosen such that IMD 10 may remain in a relatively fixed location within vasculature 12 of patient 4 once implanted. For example, vasculature 12 of patient 4 may itself hold IMD 10 in a relatively fixed location through friction or restriction.

For example, the spacing between proximal electrode 16A and distal electrode 16B may range from 5 millimeters (mm) to 55 mm, 30 mm to 55 mm, 35 mm to 55 mm, and from 40 mm to 55 mm and may be any range or individual spacing from 5 mm to 60 mm. In addition, IMD 10A may have a length L that ranges from 30 mm to about 70 mm. In other examples, the length L may range from 5 mm to 60 mm, 40 mm to 60 mm, 45 mm to 60 mm and may be any length or range of lengths between about 30 mm and about 70 mm. In addition, the width W of major surface 14 may range from 2.5 mm to 15, mm, from 2.5 mm to 10 mm, from 5 mm to 15 mm, or from 2.5 mm to 7.5 mm, and may be any single or range of widths between 2.5 mm and 15 mm. The thickness of depth D of IMD 10A may range from 2 mm to 15 mm, from 2 mm to 9 mm, from 2 mm to 5 mm, from 5 mm to 15 mm, and may be any single or range of depths between 2 mm and 15 mm. In addition, IMD 10A according to an example of the present disclosure is has a geometry and size designed for ease of implant and patient comfort. Examples of IMD 10A described in this disclosure may have a volume of three cubic centimeters (cm) or less, 1.5 cubic cm or less or any volume between three and 1.5 cubic centimeters. In the example shown in FIG. 2, proximal end 22 and distal end 23 are rounded to reduce discomfort and irritation to vasculature 12 once implanted in patient 4.

In some examples, proximal electrode 16A and distal electrode 16B may be used to sense cardiac EGM signals (e.g., ECG signals) when IMD 10 is implanted in the vasculature of patient 4. In some examples, processing circuitry of IMD 10 also may determine whether cardiac ECG signals of patient 4 are indicative of arrhythmia or other abnormalities, which processing circuitry of IMD 10 may evaluate in determining whether a medical condition (e.g., heart failure, sleep apnea, or chronic obstructive pulmonary disease (COPD)) of patient 4 has changed. The cardiac ECG signals may be stored in a memory of IMD 10, and data derived from the cardiac ECG signals, may be transmitted via integrated antenna 26 to another device, such as external device 24. Additionally, in some examples, electrodes 16A, 16B may be used by communication circuitry of IMD 10 for tissue conductance communication (TCC) communication with external device 24 or another device.

In the example shown in FIG. 2, proximal electrode 16A is in close proximity to proximal end 22, and distal electrode 16B is in close proximity to distal end 23 of IMD 10. In this example, distal electrode 16B is not limited to a flattened, outward facing surface, but may extend from first major surface 18, around rounded edges 28 or end surface 30, and onto the second major surface 20 in a three-dimensional curved configuration. As illustrated, proximal electrode 16A is located on first major surface 18 and is substantially flat and outward facing. However, in other examples not shown here, proximal electrode 16A and distal electrode 16B both may be configured like proximal electrode 16A shown in FIG. 2, or both may be configured like distal electrode 16B shown in FIG. 2. In some examples, additional electrodes 16C and 16D may be positioned on one or both of first major surface 18 and second major surface 20, such that a total of four electrodes are included on IMD 10. Any of electrodes 16A-16D may be formed of a biocompatible conductive material. For example, any of electrodes 16A-16D may be formed from any of stainless steel, titanium, platinum, iridium, or alloys thereof. In addition, electrodes of IMD 10 may be coated with a material such as titanium nitride or fractal titanium nitride, although other suitable materials and coatings for such electrodes may be used.

In the example shown in FIG. 2, proximal end 22 of IMD 10 includes header assembly 32 having one or more of proximal electrode 16A, integrated antenna 26, anti-migration projections 34, and suture hole 36. Integrated antenna 26 is located on the same major surface (e.g., first major surface 18) as proximal electrode 16A, and may be an integral part of header assembly 32. In other examples, integrated antenna 26 may be formed on the major surface opposite from proximal electrode 16A, or, in still other examples, may be incorporated within housing 15 of IMD 10. Antenna 26 may be configured to transmit or receive electromagnetic signals for communication. For example, antenna 26 may be configured to transmit to or receive signals from a programmer via inductive coupling, electromagnetic coupling, tissue conductance, Near Field Communication (NFC), Radio Frequency Identification (RFID), Bluetooth®, WiFi®, or other proprietary or non-proprietary wireless telemetry communication schemes. Antenna 26 may be coupled to communication circuitry of IMD 10, which may drive antenna 26 to transmit signals to external device 24, and may transmit signals received from external device 24 to processing circuitry of IMD 10 via communication circuitry.

In some examples, IMD 10 may include several features for retaining IMD 10 in position once implanted in vasculature 12 of patient 4, so as to decrease the chance that IMD 10 migrates in the body of patient 4. For example, as shown in FIG. 2, housing 15 may include anti-migration projections 34 positioned adjacent integrated antenna 26. Anti-migration projections 34 may include a plurality of bumps or protrusions extending away from first major surface 18, and may help prevent longitudinal movement of IMD 10 after implantation in patient 4. In other examples, anti-migration projections 34 may be located on the opposite major surface as proximal electrode 16A and/or integrated antenna 26. In addition, in the example shown in FIG. 2 header assembly 32 may include suture hole 36, which provides another means of securing IMD 10 to the patient to prevent movement following insertion. In the example shown, suture hole 36 is located adjacent to proximal electrode 16A. In some examples, header assembly 32 may include a molded header assembly made from a polymeric or plastic material, which may be integrated or separable from the main portion of IMD 10. Anchoring mechanisms like anti-migration projections 34 or suture hole 36 may be useful if IMD 10 is inserted into an artery of patient 4, rather than a vein of patient 4, or when inserted closer to the heart, rather than in the arm or leg of patient 4. For example, when IMD 10 is inserted into a vein, IMD 10 may cease to move when IMD 10 is restricted by vasculature 12 or patient 4.

In the example shown in FIG. 2, IMD 10 includes a light emitter 38, a first optical sensor 40A, and a second optical sensor 40B positioned on housing 15 of IMD 10. First optical sensor 40A may be positioned at a distance S from light emitter 38, and second optical sensor 40B may be positioned at a distance S+N from light emitter 38. In other examples, IMD 10 may include additional light emitters and/or additional optical sensors.

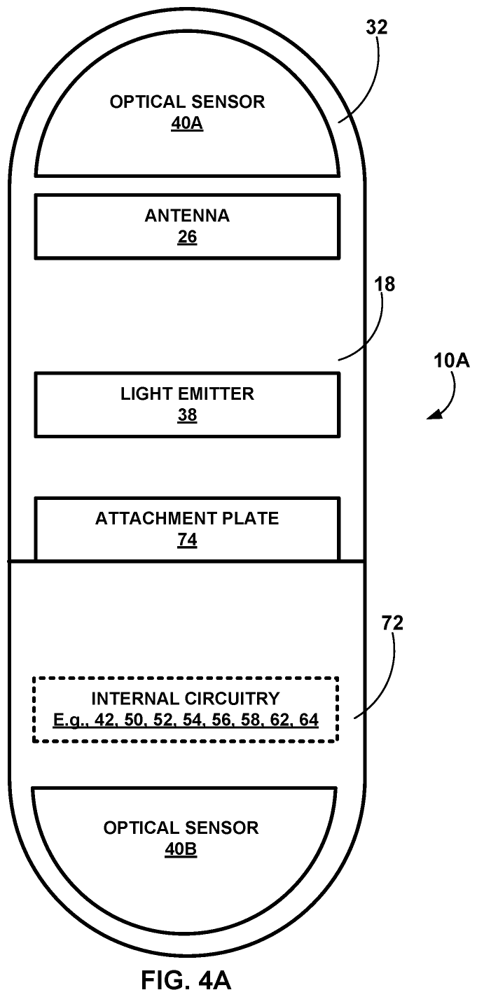

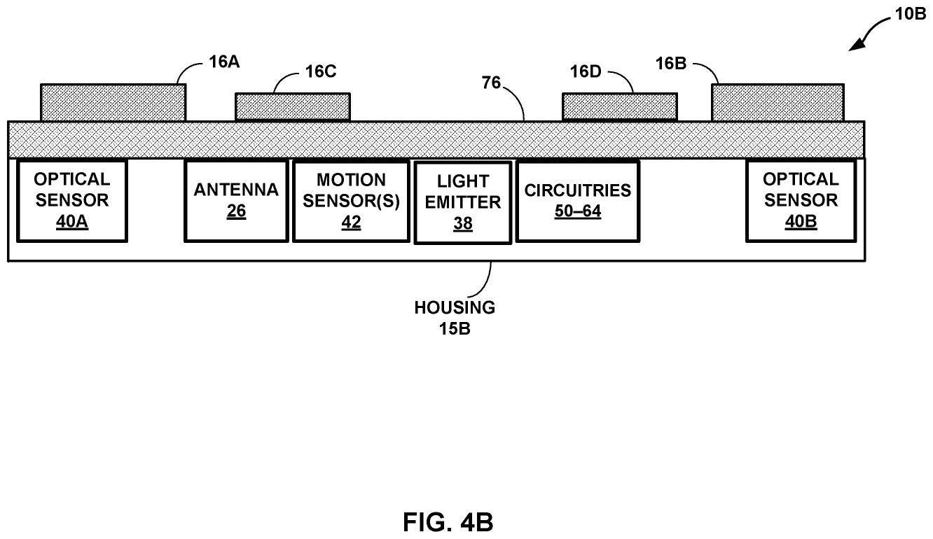

As shown in FIG. 2, light emitter 38 may be positioned on header assembly 32, although, in other examples, one or both of optical sensors 40A, 40B may additionally or alternatively be positioned on header assembly 32. In some examples, light emitter 38 may be positioned on a medial section of IMD 10, such as part way between proximal end 22 and distal end 23. Although light emitter 38 and optical sensors 40A, 40B are illustrated as being positioned on first major surface 18, light emitter 38 and optical sensors 40A, 40B alternatively may be positioned on second major surface 20. In some examples, IMD may be implanted such that light emitter 38 and optical sensors 40A, 40B face inward when IMD 10 is implanted, away from the nearest skin of patient 4, which may help minimize interference from background light coming from outside the body of patient 4. Optical sensors 40A, 40B may include a glass or sapphire window, such as described below with respect to FIG. 4B, or may be positioned beneath a portion of housing 15 of IMD 10 that is made of glass or sapphire, or otherwise transparent or translucent. In some examples, one or more of optical sensors 40A, 40B may include pulse oximetry sensors.

In some examples, IMD 10 may include one or more additional sensors, such as one or more motion sensors (e.g., accelerometers) (not shown in FIG. 2). Such motion sensor(s) may be 3D accelerometer(s) configured to generate signals indicative of one or more types of movement of the patient, such as gross body movement (e.g., motion) of the patient, patient posture, movements associated with the beating of the heart, or coughing, rales, or other respiration abnormalities, or the movement of IMD 10 within the body of patient 4. One or more of the parameters monitored by IMD 10 (e.g., cardiac flow rate, bio impedance, EGM, etc.) may fluctuate in response to changes in one or more such types of movement or posture. For example, changes in physiological parameter values, such as cardiac flow rate, sometimes may be attributable to increased patient motion (e.g., exercise or other physical motion as compared to immobility) or to changes in patient posture, and not necessarily to changes in a medical condition. Thus, in some methods of identifying or tracking a medical condition of patient 4, it may be advantageous to account for such fluctuations when determining whether a change in a parameter (e.g., cardiac flow rate) is indicative of a change in a medical condition.

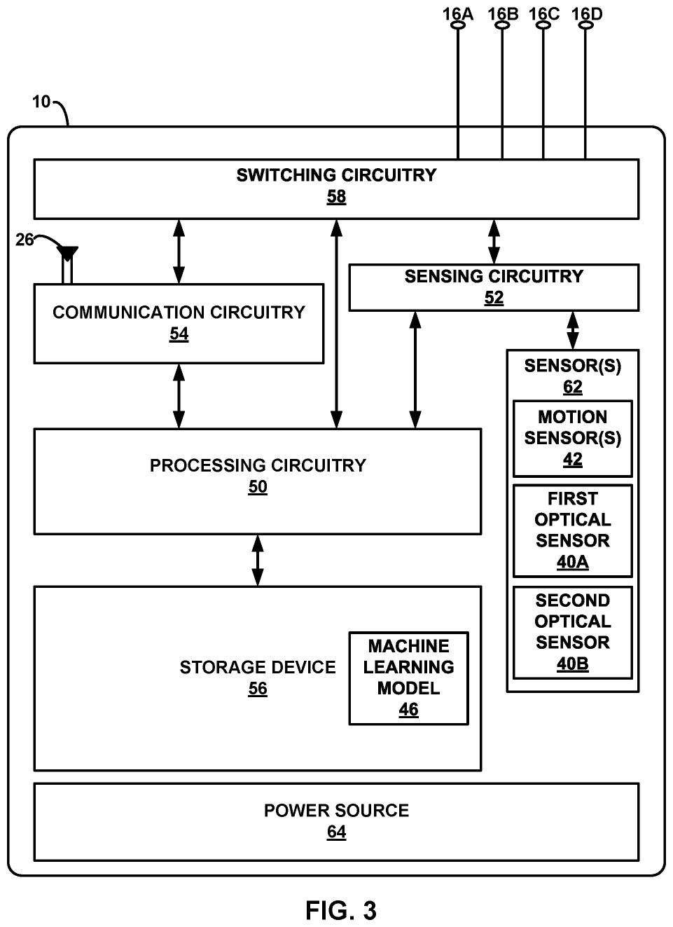

FIG. 3 is a functional block diagram illustrating an example configuration of IMD 10 of FIGS. 1 and 2, in accordance with one or more techniques described herein. In the illustrated example, IMD 10 includes electrodes 16, antenna 26, processing circuitry 50, sensing circuitry 52, communication circuitry 54, storage device 56, switching circuitry 58, sensors 62 including motion sensor(s) 42 (which may be an accelerometer), first optical sensor 40A, second optical sensor 40B, and power source 64. First optical sensor 40A and second optical sensor 40B may be displaced from each other longitudinally along the length of IMD 10. Processing circuitry 50 may include fixed function circuitry and/or programmable processing circuitry. Processing circuitry 50 may include any one or more of a microprocessor, a controller, a DSP, an ASIC, an FPGA, or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof. In some examples, one or more techniques of this disclosure may be performed by processing circuitry 50.

Sensing circuitry 52 and communication circuitry 54 may be selectively coupled to electrodes 16A-16D via switching circuitry 58, as controlled by processing circuitry 50. Sensing circuitry 52 may monitor signals from electrodes 16A-16D in order to monitor electrical activity of heart (e.g., to produce an ECG). Sensing circuitry 52 also may monitor signals from sensors 62, which may include motion sensor(s) 42 (which may be an accelerometer), first optical sensor 40A, second optical sensor 40B, and any additional optical sensors that may be positioned on IMD 10. In some examples, sensing circuitry 52 may include one or more filters and amplifiers for filtering and amplifying signals received from one or more of electrodes 16A-16D and/or motion sensor(s) 42 (which may be an accelerometer).

Communication circuitry 54 may include any suitable hardware, firmware, software or any combination thereof for communicating with another device, such as external device 24 or another IMD or sensor, such as a pressure sensing device. Under the control of processing circuitry 50, communication circuitry 54 may receive downlink telemetry from, as well as send uplink telemetry to, external device 24 or another device with the aid of an internal or external antenna, e.g., antenna 26. In addition, processing circuitry 50 may communicate with a networked computing device via an external device (e.g., external device 24) and a computer network, such as the Medtronic CareLink® Network developed by Medtronic, Inc, of Minneapolis, Minnesota.

A clinician or other user may retrieve data from IMD 10 using external device 24, or by using another local or networked computing device configured to communicate with processing circuitry 50 via communication circuitry 54. The clinician may also program parameters of IMD 10 using external device 24 or another local or networked computing device.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media, such as a random access memory (RAM), ferroelectric RAM (FRAM) read-only memory (ROM), non-volatile RAM (NVRAM), electrically-erasable programmable ROM (EEPROM), flash memory, or any other digital media.

Storage device 56 may also store machine learning model 46. In some examples, machine learning model 46 may be used by processing circuitry 50 to determine whether to generate an indication for output as discussed above.

Power source 64 is configured to deliver operating power to the components of IMD 10. Power source 64 may include a battery and a power generation circuit to produce the operating power. In some examples, the battery is rechargeable to allow extended operation. In some examples, recharging is accomplished through proximal inductive interaction between an external charger and an inductive charging coil within external device 24. Power source 64 may include any one or more of a plurality of different battery types, such as nickel cadmium batteries and lithium ion batteries. A non-rechargeable battery may be selected to last for several years, while a rechargeable battery may be inductively charged from an external device, e.g., on a daily or weekly basis.

FIGS. 4A and 4B illustrate two additional example IMDs that may be substantially similar to IMD 10 of FIGS. 1-3, but which may include one or more additional features, in accordance with one or more techniques described herein. In the example of FIGS. 4A-4B, IMD 10A and IMD 10B do not show electrodes, but electrodes may be included. As shown in FIGS. 4A-4B, optical sensors 40A and 40B may be respectively located near each longitudinal end of IMD 10A and IMD 10B which may increase the distance between optical sensors 40A and 40B when compared to the example of FIG. 2 (e.g., the distance “N”). Locating optical sensors 40A and 40B near each longitudinal end of IMD 10A or IMD 10B may improve accuracy of any determined cardiac flow rate. The components of FIGS. 4A and 4B may not necessarily be drawn to scale, but instead may be enlarged to show detail. FIG. 4A is a block diagram of a top view of an example configuration of an IMD 10A. FIG. 4B is a block diagram of a side view of example IMD 10B, which may include an insulative layer as described below.

FIG. 4A is a conceptual drawing illustrating another example IMD 10A that may be substantially similar to IMD 10 of FIG. 1. In addition to the components illustrated in FIGS. 1-3, the example of IMD 10 illustrated in FIG. 4A also may include a body portion 72 and an attachment plate 74. Attachment plate 74 may be configured to mechanically couple header assembly 32 to body portion 72 of IMD 10A. Body portion 72 of IMD 10A may be configured to house one or more of the internal components of IMD 10 illustrated in FIG. 3, such as one or more of processing circuitry 50, sensing circuitry 52, communication circuitry 54, storage device 56, switching circuitry 58, internal components of sensors 62, and power source 64. In some examples, body portion 72 may be formed of one or more of titanium, ceramic, or any other suitable biocompatible materials.

FIG. 4B is a conceptual drawing illustrating another example IMD 10B that may include components substantially similar to IMD 10 of FIG. 1. In addition to the components illustrated in FIGS. 1-3, the example of IMD 10B illustrated in FIG. 4B also may include a wafer-scale insulative cover 76, which may help insulate electrical signals passing between electrodes 16A-16D and/or optical sensors 40A, 40B on housing 15B and processing circuitry 50. In some examples, insulative cover 76 may be positioned over an open housing 15 to form the housing for the components of IMD 10B. One or more components of IMD 10B (e.g., antenna 26, light emitter 38, optical sensors 40A, 40B, processing circuitry 50, sensing circuitry 52, communication circuitry 54, switching circuitry 58, and/or power source 64) may be formed on a bottom side of insulative cover 76, such as by using flip-chip technology. Insulative cover 76 may be flipped onto a housing 15B.

Insulative cover 76 may be configured so as not to interfere with the operation of IMD 10B. For example, one or more of electrodes 16A-16D may be formed or placed above or on top of insulative cover 76, and electrically connected to switching circuitry 58 through one or more vias (not shown) formed through insulative cover 76. Insulative cover 76 may be formed of sapphire (i.e., corundum), glass, parylene, and/or any other suitable insulating material. Sapphire may be greater than 80% transmissive for wavelengths in the range of about 300 nm to about 4000 nm, and may have a relatively flat profile. In the case of variation, different transmissions at different wavelengths may be compensated for, such as by using a ratiometric approach. Housing 15B may be formed from titanium or any other suitable material (e.g., a biocompatible material), and may have a thickness of about 200 micrometers to about 500 micrometers. These materials and dimensions are examples only, and other materials and other thicknesses are possible for devices of this disclosure.

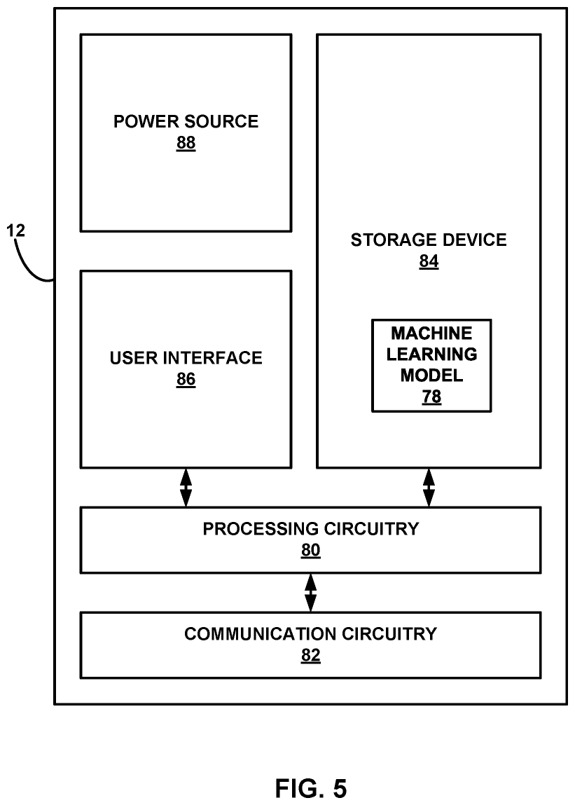

FIG. 5 is a block diagram illustrating an example configuration of components of external device 24, in accordance with one or more techniques of this disclosure. In the example of FIG. 5, external device 24 includes processing circuitry 80, communication circuitry 82, storage device 84, user interface 86, and power source 88.

Processing circuitry 80, in one example, may include one or more processors that are configured to implement functionality and/or process instructions for execution within external device 24. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80. In some examples, processing circuitry 80 may perform one or more of the techniques of this disclosure.

Communication circuitry 82 may include any suitable hardware, firmware, software or any combination thereof for communicating with another device, such as IMD 10. Under the control of processing circuitry 80, communication circuitry 82 may receive downlink telemetry from, as well as send uplink telemetry to, IMD 10, or another device.

Storage device 84 may be configured to store information within external device 24 during operation. Storage device 84 may include a computer-readable storage medium or computer-readable storage device. In some examples, storage device 84 includes one or more of a short-term memory or a long-term memory. Storage device 84 may include, for example, RAM, dynamic random access memories (DRAM), static random access memories (SRAM), magnetic discs, optical discs, flash memories, or forms of electrically programmable memories (EPROM) or EEPROM. In some examples, storage device 84 is used to store data indicative of instructions for execution by processing circuitry 80. Storage device 84 may be used by software or applications running on external device 24 to temporarily store information during program execution.

Storage device 84 may also store machine learning model 78. In some examples, machine learning model 78 may be used by processing circuitry 80 to determine whether to generate an indication for output as discussed herein. In the example where machine learning model 78 is located in storage device 84, external device 24 may generate the indication for output to another external device such as a server, e.g., server 94 of FIG. 6.

Data exchanged between external device 24 and IMD 10 may include operational parameters. External device 24 may transmit data including computer readable instructions which, when implemented by IMD 10, may control IMD 10 to change one or more operational parameters and/or export collected data. For example, processing circuitry 80 may transmit an instruction to IMD 10 which requests IMD 10 to export collected data (e.g., data corresponding to one or more of a cardiac flow rate, optical sensor signals, an accelerometer signal, or other collected data) to external device 24. In turn, external device 24 may receive the collected data from IMD 10 and store the collected data in storage device 84. Additionally, or alternatively, processing circuitry 80 may export instructions to IMD 10 requesting IMD 10 to update one or more operational parameters of IMD 10.

A user, such as a clinician, patient 4, or a caregiver, may interact with external device 24 through user interface 86. User interface 86 includes a display (not shown), such as an LCD or LED display or other type of screen, with which processing circuitry 80 may present information related to IMD 10 (e.g., a determined cardiac flow rate or other sensed physiological parameters). In addition, user interface 86 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 24 and provide input. In other examples, user interface 86 also includes audio circuitry for providing audible notifications, instructions or other sounds to patient 4, receiving voice commands from patient 4, or both. Storage device 84 may include instructions for operating user interface 86 and for managing power source 88.

Power source 88 is configured to deliver operating power to the components of external device 24. Power source 88 may include a battery and a power generation circuit to produce the operating power. In some examples, the battery is rechargeable to allow extended operation. Recharging may be accomplished by electrically coupling power source 88 to a cradle or plug that is connected to an alternating current (AC) outlet. In addition, recharging may be accomplished through proximal inductive interaction between an external charger and an inductive charging coil within external device 24. In other examples, traditional batteries (e.g., nickel cadmium or lithium ion batteries) may be used. In addition, external device 24 may be directly coupled to an alternating current outlet to operate.

FIG. 6 is a block diagram illustrating an example system that includes an access point 90, a network 92, external computing devices, such as a server 94, and one or more other computing devices 100A-100N, which may be coupled to IMD 10, external device 24, and processing circuitry 14 via network 92, in accordance with one or more techniques described herein. In this example, IMD 10 may use communication circuitry 54 to communicate with external device 24 via a first wireless connection, and to communication with an access point 90 via a second wireless connection. In the example of FIG. 6, access point 90, external device 24, server 94, and computing devices 100A-100N are interconnected and may communicate with each other through network 92.

Access point 90 may include a device that connects to network 92 via any of a variety of connections, such as telephone dial-up, digital subscriber line (DSL), fiber optic, or cable modem connections. In other examples, access point 90 may be coupled to network 92 through different forms of connections, including wired or wireless connections. In some examples, access point 90 may be a user device, such as a tablet or smartphone, that may be co-located with the patient. As discussed above, IMD 10 may be configured to transmit data, such as cardiac flow rate of patient 4, optical sensor signals, an accelerometer signal, or other data collected by IMD 10 to external device 24. In addition, access point 90 may interrogate IMD 10, such as periodically or in response to a command from the patient or network 92, in order to retrieve parameter values determined by processing circuitry 50 of IMD 10, or other operational or patient data from IMD 10. Access point 90 may then communicate the retrieved data to server 94 via network 92.

In some cases, server 94 may be configured to provide a secure storage site for data that has been collected from IMD 10, and/or external device 24, such as cardiac flow rates or averages thereof of patient 4. In some cases, server 94 may assemble data in web pages or other documents for viewing by trained professionals, such as clinicians, via computing devices 100A-100N. One or more aspects of the illustrated system of FIG. 6 may be implemented with general network technology and functionality, which may be similar to that provided by the Medtronic CareLink® Network developed by Medtronic plc, of Dublin, Ireland.

Server 94 may include processing circuitry 96. Processing circuitry 96 may include fixed function circuitry and/or programmable processing circuitry. Processing circuitry 96 may include any one or more of a microprocessor, a controller, a DSP, an ASIC, an FPGA, or equivalent discrete or analog logic circuitry. In some examples, processing circuitry 96 may include multiple components, such as any combination of one or more microprocessors, one or more controllers, one or more DSPs, one or more ASICs, or one or more FPGAs, as well as other discrete or integrated logic circuitry. The functions attributed to processing circuitry 96 herein may be embodied as software, firmware, hardware or any combination thereof. In some examples, processing circuitry 96 may perform one or more techniques described herein. For example, processing circuitry 96 may determine cardiac flow rate based on signals from first optical sensor 40A and second optical sensor 40B (FIGS. 2-4B) collected by IMD 10.

Server 94 may include memory 98. Memory 98 includes computer-readable instructions that, when executed by processing circuitry 96, cause IMD 10 and processing circuitry 96 to perform various functions attributed to IMD 10 and processing circuitry 96 herein. Memory 98 may include any volatile, non-volatile, magnetic, optical, or electrical media, such as RAM, ROM, NVRAM, EEPROM, flash memory, or any other digital media. In some examples, memory 98 may store a machine learning model used by processing circuitry 96 as described herein.

In some examples, one or more of computing devices 100A-100N (e.g., device 100A) may be a tablet or other smart device located with a clinician, by which the clinician may program, receive alerts from, and/or interrogate IMD 10. For example, the clinician may access data corresponding to cardiac flow rates of patient 4 determined by IMD 10, external device 24, processing circuitry 14, or server 94 through device 100A, such as when patient 4 is in between clinician visits, to check on a status of a medical condition, such as heart failure. In some examples, the clinician may enter instructions for a medical intervention for patient 4 into an app in device 100A, such as based on a status of a patient condition determined by IMD 10, external device 24, processing circuitry 14, or any combination thereof, or based on other patient data known to the clinician. Device 100A then may transmit the instructions for medical intervention to another of computing devices 100A-100N (e.g., device 100B) located with patient 4 or a caregiver of patient 4. For example, such instructions for medical intervention may include an instruction to change a drug dosage, timing, or selection, to schedule a visit with the clinician, or to seek medical attention. In further examples, device 100B may generate an indication for output, such as an alert to patient 4 based on a status of a medical condition of patient 4 determined by IMD 10, which may enable patient 4 proactively to seek medical attention prior to receiving instructions for a medical intervention. In this manner, patient 4 may be empowered to take action, as needed, to address his or her medical status, which may help improve clinical outcomes for patient 4.

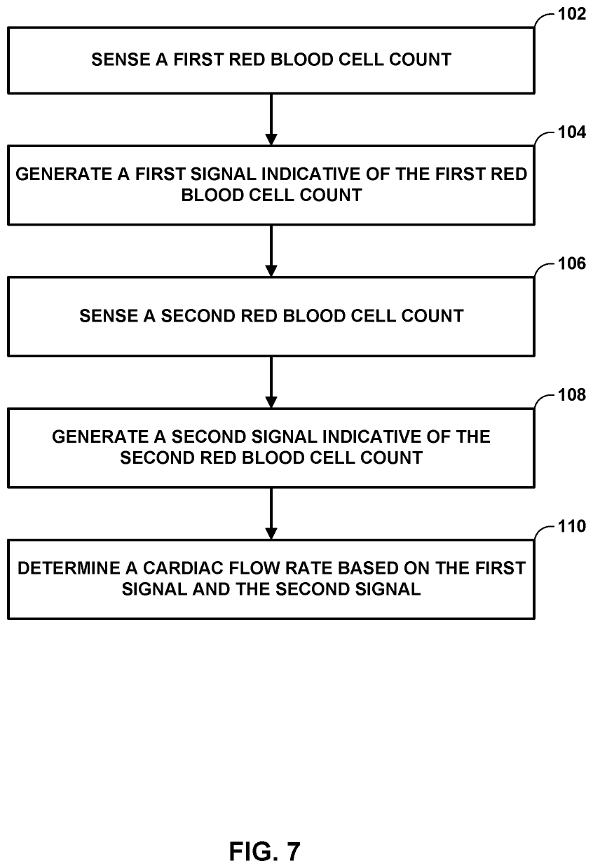

FIG. 7 is a flow diagram illustrating an example of cardiac flow rate determination techniques according to one or more aspects of this disclosure. The example of FIG. 7 is discussed with respect to IMD 10 of FIG. 3, but these techniques may be implemented in any combination of devices of system 2 (FIG. 1) or of FIG. 6, e.g., by processing circuitry of any one or more devices described herein.

First optical sensor 40A may sense a first red blood cell count (102). For example, first optical sensor may sense light from light emitter 38 (FIG. 2) which may be reflected of red blood cells above first optical sensor 40A within vasculature 12 of patient 4. First optical sensor 40A may generate a first signal indicative of the first red blood cell count (104). For example, first optical sensor 40A may generate an electrical or an optical signal indicative of the first red blood cell count. First optical sensor 40A may be disposed on a first portion of a medical device (e.g., IMD 10) having an elongated housing and being configured to be at least substantially entirely inserted within vasculature 12 of patient 4. In some examples, at least substantially entirely inserted within vasculature 12 of patient 4 means at least 80% of the medical device may be inserted within vasculature 12 of patient 4. In some examples, at least substantially entirely inserted within vasculature 12 of patient 4 means at least 95% of the medical device may be inserted within vasculature 12 of patient 4. In other examples, at least substantially entirely inserted within vasculature 12 of patient 4 means 100% of the medical device may be inserted within vasculature 12 of patient 4.

Second optical sensor 40B may sense a second red blood cell count (106). For example, second optical sensor 40B may sense light from light emitter 38 (FIG. 2) which may be reflected of red blood cells above second optical sensor 40B within vasculature 12 of patient 4. Second optical sensor 40B may generate a second signal indicative of the second red blood cell count (108). For example, second optical sensor 40B may generate an electrical or an optical signal indicative of the first red blood cell count. Second optical sensor 40B may be disposed on a second portion of medical device (e.g., IMD 10), the second portion of the device being longitudinally displaced from the first portion of the medical device.

Processing circuitry 50 may determine a cardiac flow rate based on a first signal and the second signal (110). For example, processing circuitry may determine the first red blood cell count from the first signal and determine the second red blood cell count from the second signal. Processing circuitry 50 may determine a length of time from when the first red blood cell count equals or is within a predetermined buffer amount of being equal to the second red blood cell count. Processing circuitry 50 may determine the cardiac flow rate based at least in part on the determined length of time and a distance between the first optical sensor and the second optical sensor.

In some examples, processing circuitry may determine a speed of red blood cells based on the determined length of time and the distance between the first optical sensor and the second optical sensor and apply a formula of flow rates at a given pressurized tube state to the determined speed of the red blood cells. In some examples, at least one of first optical sensor 40A or second optical sensor 40B is a pulse oximetry sensor. In some examples, IMD 10 includes one or more motion sensors (e.g., motion sensor(s) 42) configured to generate at least one motion signal indicative of a posture or activity of the patient. In some examples, processing circuitry 50 may determine a plurality of postures or activities of the patient based on the at least one motion signal and determine at least respective one cardiac flow rate for each of the plurality of postures or activities of the patient.

In some examples, communication circuitry 54 may transmit the determined cardiac flow rate to an external device (e.g., external device 24). In some examples, processing circuitry 50 may use a machine learning model and the determined cardiac flow rate to determine whether to generate an indication for output to an external device (e.g., external device 24). In some examples, the processing circuitry, e.g., of IMD 10, external device 24, or server 94, may apply one or more determined cardiac flow rates, e.g., for one or more postures or activity levels, to a machine learning model to determine one or more output values indicating the likelihood or degree of one or more health events occurring presently or at a future time. In addition to determined flow rates, inputs to the machine learning model may include activity level, posture, disease state, comorbidities, or any other physiological or other parameter of the patient described herein. The processing circuitry may compare the one or more values to one or more thresholds or other criteria to determine whether to transmit the indication.

In some examples, communication circuitry 54 may transmit the indication to the external device, wherein the indication comprises at least one of: an alert to seek medical attention; an alert comprising medical instructions for the patient; or an alert for a clinician. In some examples, the elongated housing includes an anti-clotting coating, such as a steroidal coating. In some examples, IMD 10 includes an anchoring mechanism (e.g., anti-migration projections 34 or suture hole 36 (FIG. 2).

In some examples, a medical device (e.g., IMD 10) is configured to be at least substantially entirely inserted into a vasculature of a patient. In some examples, the medical device includes an elongated housing; a first sensor configured to sense a first vascular particle count and generate a first signal indicative of the first vascular particle count, the first sensor being disposed on a first portion of the medical device and a second sensor configured to sense a second vascular particle count and generate a second signal indicative of the second vascular particle count, the second sensor being disposed on a second portion of the medical device, the second portion of the medical device being longitudinally displaced from the first portion of the medical device. In some examples, the medical device includes processing circuitry configured to determine a cardiac flow rate based on a first signal and the second signal. In some examples, the first vascular particle and the second vascular particle comprise at least one of red blood cells, white blood cells, or platelets.

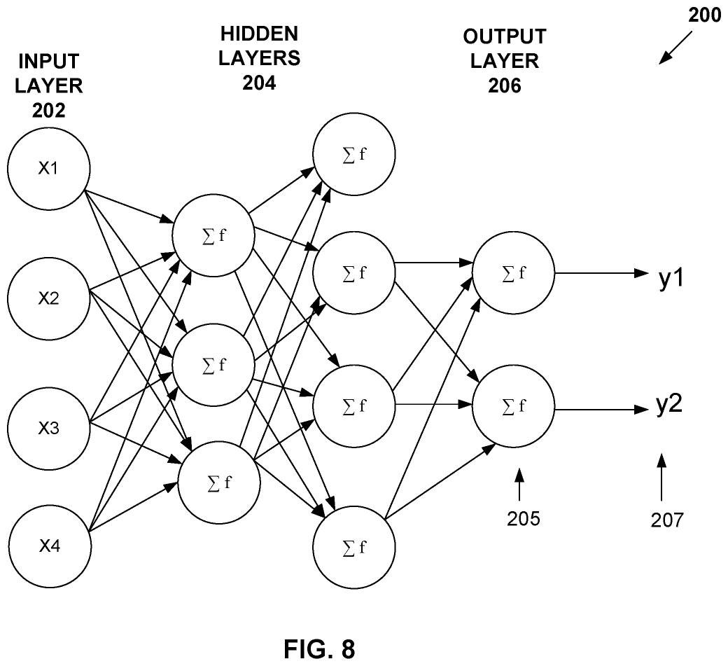

FIG. 8 is a conceptual diagram illustrating an example machine learning model 200 configured to determine one or more output values based on cardiac flow rate data. Machine learning model 200 may correspond to machine learning models 46 and 78. Machine learning model 200 is an example of a deep learning model, or deep learning algorithm, trained to determine whether a particular set of patient parameter data indicates a condition of the patient meriting a system communication as described herein. One or more of IMD 10, external device 24, or server 94 may train, store, and/or utilize machine learning model 200, but other devices may apply inputs associated with a particular patient to machine learning model 200 in other examples. Some non-limiting examples of machine learning techniques include Support Vector Machines, K-Nearest Neighbor algorithm, and Multi-layer Perceptron.

As shown in the example of FIG. 8, machine learning model 200 may include three layers. These three layers include input layer 202, hidden layer 204, and output layer 206. Output layer 206 comprises the output from the transfer function 205 of output layer 206. Input layer 202 represents each of the input values X1 through X4 provided to machine learning model 200. The number of inputs may be less than or greater than 4, including much greater than 4, e.g., hundreds or thousands. In some examples, the input values may any of the of values input into a machine learning model, as described above. In some examples, input values may include one or more cardiac flow rate values. In addition, in some examples input values of machine learning model 200 may include additional data, such as data relating to one or more additional parameters of patient 4.

Each of the input values for each node in the input layer 202 is provided to each node of hidden layer 204. In the example of FIG. 8, hidden layers 204 include two layers, one layer having four nodes and the other layer having three nodes, but fewer or greater number of nodes may be used in other examples. Each input from input layer 202 is multiplied by a weight and then summed at each node of hidden layers 204. During training of machine learning model 200, the weights for each input are adjusted to establish the relationship between the inputs determining whether a particular set of inputs represents a health event and/or determining a score indicative of whether a set of inputs may be representative of a health event. In some examples, one hidden layer may be incorporated into machine learning model 200, or three or more hidden layers may be incorporated into machine learning model 200, where each layer includes the same or different number of nodes.

The result of each node within hidden layers 204 is applied to the transfer function of output layer 206. The transfer function may be liner or non-linear, depending on the number of layers within machine learning model 200. Example non-linear transfer functions may be a sigmoid function or a rectifier function. The output 207 of the transfer function may be a classification that indicates whether the particular set of cardiac flow rates or other input set represents a health event and/or a score indicative of an extent to which the input data set represents a health event. By applying the cardiac flow rate data and/or other patient parameter data to a machine learning model, such as machine learning model 200, processing circuitry of system 2 is able to determine a patient is experiencing or will soon experience a health event with great accuracy, specificity, and sensitivity. This may facilitate alerts and other interventions as described herein.

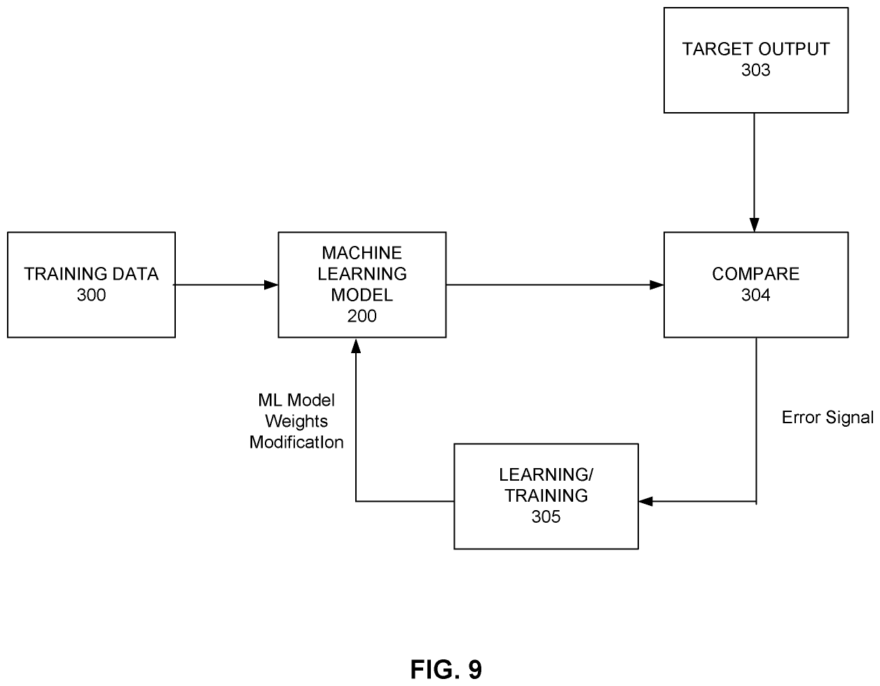

FIG. 9 is an example of a machine learning model 200 being trained using supervised and/or reinforcement learning techniques. Machine learning model 200 may be implemented using any number of models for supervised and/or reinforcement learning, such as but not limited to, an artificial neural network, a decision tree, naïve Bayes network, support vector machine, or k-nearest neighbor model, to name only a few of the examples discussed above. In some examples, processing circuitry one or more of IMD 10, external device 24, and/or server 94 initially trains the machine learning model 200 based on training set data 300 including numerous instances of input data corresponding to health events and non-health events, e.g., as labeled by an expert. A prediction or classification by the machine learning model 200 may be compared 304 to the target output 303, e.g., as determined based on the label. Based on an error signal representing the comparison, the processing circuitry implementing a learning/training function 305 may send or apply a modification to weights of machine learning model 200 or otherwise modify/update the machine learning model 200. For example, one or more of IMD 10, external device 24, and/or server 94 may, for each training instance in the training set 300, modify machine learning model 200 to change a score generated by the machine learning model 200 in response to data applied to the machine learning model 200. This disclosure includes the following non-limiting examples.

Example 1. A medical device configured to be as least substantially entirely inserted into a vasculature of a patient, the medical device comprising: an elongated housing; a first optical sensor configured to sense a first red blood cell count and generate a first signal indicative of the first red blood cell count, the first optical sensor being disposed on a first portion of the medical device; a second optical sensor configured to sense a second red blood cell count and generate a second signal indicative of the second red blood cell count, the second optical sensor being disposed on a second portion of the medical device, the second portion of the medical device being longitudinally displaced from the first portion of the medical device; and processing circuitry configured to determine a cardiac flow rate based on a first signal and the second signal.

Example 2. The medical device of claim 1, wherein as part of determining the cardiac flow rate, the processing circuitry is configured to: determine the first red blood cell count from the first signal; determine the second red blood cell count from the second signal; determine a length of time from when the first red blood cell count equals or is within a predetermined buffer amount of being equal to the second red blood cell count; and determine the cardiac flow rate based at least in part on the determined length of time and a distance between the first optical sensor and the second optical sensor.

Example 3. The medical device of claim 2, wherein as part of determining the cardiac flow rate, the processing circuitry is further configured to: determine a speed of red blood cells based on the determined length of time and the distance between the first optical sensor and the second optical sensor; and apply a formula of flow rates at a given pressurized tube state to the determined speed of the red blood cells.

Example 4. The medical device of any of claims 1-3, wherein at least one of the first optical sensor or the second optical sensor is a pulse oximetry sensor.

Example 5. The medical device of any of claims 1-4, further comprising one or more motion sensors configured to generate at least one motion signal indicative of a posture or activity of the patient, and wherein the processing circuitry is further configured to: determine a plurality of postures or activities of the patient based on the at least one motion signal; and determine at least respective one cardiac flow rate for each of the plurality of postures or activities of the patient.

Example 6. The medical device of any of claims 1-5, further comprising telemetry circuitry configured to transmit the determined cardiac flow rate to an external device.

Example 7. The medical device of claim 6, further comprising a machine learning algorithm and wherein the processing circuitry is further configured to: use the machine learning algorithm and the determined cardiac flow rate to determine whether to generate an indication for output to an external device.

Example 8. The medical device of claim 7, wherein the telemetry circuitry is further configured to transmit the indication to the external device, wherein the indication comprises at least one of: an alert to seek medical attention; an alert comprising medical instructions for the patient; or an alert for a clinician.

Example 9. The medical device of any of claims 1-8, wherein the

elongated housing comprises an anti-clotting coating.

Example 10. The medical device of any of claims 1-9, further comprising an anchoring mechanism configured to anchor the medical device within the vasculature of the patient.

Example 11. A method comprising: sensing, by a first optical sensor, a first red blood cell count; generating, by the first optical sensor, a first signal indicative of the first red blood cell count, the first optical sensor being disposed on a first portion of a medical device having an elongated housing and being configured to be at least substantially entirely inserted within a vasculature of a patient; sensing, by a second optical sensor, a second red blood cell count; generating, by the second optical sensor, a second signal indicative of the second red blood cell count, the second optical sensor being disposed on a second portion of the medical device, the second portion of the medical device being longitudinally displaced from the first portion of the medical device; and determining, by processing circuitry, a cardiac flow rate based on a first signal and the second signal.

Example 12. The method of claim 11, wherein determining the cardiac flow rate comprises: determine the first red blood cell count from the first signal; determine the second red blood cell count from the second signal; determine a length of time from when the first red blood cell count equals or is within a predetermined buffer amount of being equal to the second red blood cell count; and determine the cardiac flow rate based at least in part on the determined length of time and a distance between the first optical sensor and the second optical sensor.

Example 13. The method of claim 12, wherein determining the cardiac flow rate further comprises: determining a speed of red blood cells based on the determined length of time and the distance between the first optical sensor and the second optical sensor; and applying a formula of flow rates at a given pressurized tube state to the determined speed of the red blood cells.

Example 14. The method of any of claims 11-13, wherein at least one of the first optical sensor or the second optical sensor is a pulse oximetry sensor.

Example 15. The method of any of claims 11-14, further comprising one or more motion sensors configured to generate at least one motion signal indicative of a posture or activity of the patient, the method further comprising: determining a plurality of postures or activities of the patient based on the at least one motion signal; and determining at least respective one cardiac flow rate for each of the plurality of postures or activities of the patient.

Example 16. The method of any of claims 11-15, further comprising transmitting, by telemetry circuitry, the determined cardiac flow rate to an external device.

Example 17. The method of any of claims 11-16, further comprising using, by the processing circuitry, a machine learning algorithm and the determined cardiac flow rate to determine whether to generate an indication for output to an external device.

Example 18. The method of claim 17, further comprising transmitting the indication to the external device, wherein the indication comprises at least one of: an alert to seek medical attention; an alert comprising medical instructions for the patient; or an alert for a clinician.

Example 19. The method of any of claims 11-18, wherein the elongated housing comprises an anti-clotting coating.

Example 20. A non-transitory computer-readable storage medium storing instructions, which when executed, cause processing circuitry to: determine a cardiac flow rate based on a first signal and a second signal, wherein the first signal is from a first optical sensor disposed on a first portion of a medical device having an elongated housing and being configured to be at least substantially entirely inserted within a vasculature of a patient, the first signal being indicative of a first red blood cell count, and wherein the second signal is from a second optical sensor disposed on a second portion of the medical device, the second signal being indicative of a second red blood cell count, the second portion of the medical device being longitudinally displaced from the first portion of the medical device.

Example 21. A medical device configured to be at least substantially entirely inserted into a vasculature of a patient, the medical device comprising: an elongated housing; a first sensor configured to sense a first vascular particle count and generate a first signal indicative of the first vascular particle count, the first sensor being disposed on a first portion of the medical device; a second sensor configured to sense a second vascular particle count and generate a second signal indicative of the second vascular particle count, the second sensor being disposed on a second portion of the medical device, the second portion of the medical device being longitudinally displaced from the first portion of the medical device; and processing circuitry configured to determine a cardiac flow rate based on a first signal and the second signal.

Example 22. The medical device of example 21, wherein the first vascular particle and the second vascular particle comprise at least one of red blood cells, white blood cells, or platelets.

The techniques described in this disclosure may be implemented, at least in part, in hardware, software, firmware, or any combination thereof. For example, various aspects of the techniques may be implemented within one or more microprocessors, DSPs, ASICS, FPGAs, or any other equivalent integrated or discrete logic QRS circuitry, as well as any combinations of such components, embodied in external devices, such as clinician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, FRAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

Various examples have been described. These and other examples are within the scope of the following claims.

1. A medical device comprising:
an elongated housing configured to be at least substantially entirely inserted into a vasculature of a patient;
a first optical sensor configured to sense a first red blood cell count and generate a first signal indicative of the first red blood cell count, the first optical sensor being disposed on or within a first portion of the housing;
a second optical sensor configured to sense a second red blood cell count and generate a second signal indicative of the second red blood cell count, the second optical sensor being disposed on or within a second portion of the housing, the second portion of the housing being longitudinally displaced from the first portion of the housing; and
processing circuitry within the housing, the processing circuitry configured to determine a cardiac flow rate based on a first signal and the second signal.

2. The medical device of claim 1, wherein as part of determining the cardiac flow rate, the processing circuitry is configured to:
determine the first red blood cell count from the first signal;
determine the second red blood cell count from the second signal;
determine a length of time from when the first red blood cell count equals or is within a predetermined buffer amount of being equal to the second red blood cell count; and
determine the cardiac flow rate based at least in part on the determined length of time and a distance between the first optical sensor and the second optical sensor.

3. The medical device of claim 2, wherein as part of determining the cardiac flow rate, the processing circuitry is further configured to:
determine a speed of red blood cells based on the determined length of time and the distance between the first optical sensor and the second optical sensor; and
apply a formula of flow rates at a given pressurized tube state to the determined speed of the red blood cells.

4. The medical device of claim 1, wherein at least one of the first optical sensor or the second optical sensor is a pulse oximetry sensor.

5. The medical device of claim 1, further comprising one or more motion sensors configured to generate at least one motion signal indicative of a posture or activity of the patient, and wherein the processing circuitry is further configured to:
determine a plurality of postures or activities of the patient based on the at least one motion signal; and
determine at least one respective cardiac flow rate for each of the plurality of postures or activities of the patient.

6. The medical device of claim 1, further comprising telemetry circuitry configured to transmit the determined cardiac flow rate to an external device.

7. The medical device of claim 6, wherein the processing circuitry is further configured to apply the determined cardiac flow rate to a machine learning model to determine whether to generate an indication for output to an external device.

8. The medical device of claim 7, wherein the telemetry circuitry is further configured to transmit the indication to the external device, wherein the indication comprises at least one of:
an alert to seek medical attention;
an alert comprising medical instructions for the patient; or an alert for a clinician.

9. The medical device of claim 1, wherein the elongated housing comprises an anti-clotting coating.

10. The medical device of claim 1, further comprising an anchoring mechanism configured to anchor the medical device within the vasculature of the patient.

11-13. (canceled)

14. A method comprising:
sensing, by a first optical sensor, a first red blood cell count;
generating, by the first optical sensor, a first signal indicative of the first red blood cell count, the first optical sensor being disposed on a first portion of a medical device having an elongated housing and being configured to be at least substantially entirely inserted within a vasculature of a patient;
sensing, by a second optical sensor, a second red blood cell count;
generating, by the second optical sensor, a second signal indicative of the second red blood cell count, the second optical sensor being disposed on a second portion of the medical device, the second portion of the medical device being longitudinally displaced from the first portion of the medical device; and
determining, by processing circuitry, a cardiac flow rate based on a first signal and the second signal.

15. The method of claim 14, wherein determining the cardiac flow rate comprises:
determine the first red blood cell count from the first signal;
determine the second red blood cell count from the second signal;
determine a length of time from when the first red blood cell count equals or is within a predetermined buffer amount of being equal to the second red blood cell count; and
determine the cardiac flow rate based at least in part on the determined length of time and a distance between the first optical sensor and the second optical sensor.

16. The method of claim 15, wherein determining the cardiac flow rate further comprises:
determining a speed of red blood cells based on the determined length of time and the distance between the first optical sensor and the second optical sensor; and
applying a formula of flow rates at a given pressurized tube state to the determined speed of the red blood cells.

17. The method of claim 14, wherein at least one of the first optical sensor or the second optical sensor is a pulse oximetry sensor.

18. The method of claim 14, further comprising one or more motion sensors configured to generate at least one motion signal indicative of a posture or activity of the patient, the method further comprising:
determining a plurality of postures or activities of the patient based on the at least one motion signal; and
determining at least respective one cardiac flow rate for each of the plurality of postures or activities of the patient.

19. The method of claim 14, further comprising transmitting, by telemetry circuitry, the determined cardiac flow rate to an external device.

20. The method of claim 14, further comprising using, by the processing circuitry, a machine learning algorithm and the determined cardiac flow rate to determine whether to generate an indication for output to an external device.

21. The method of claim 20, further comprising transmitting the indication to the external device, wherein the indication comprises at least one of:
an alert to seek medical attention;
an alert comprising medical instructions for the patient; or
an alert for a clinician.

22. The method of claim 11, wherein the elongated housing comprises an anti-clotting coating.

23. A non-transitory computer-readable storage medium storing instructions, which when executed, cause processing circuitry to:
determine a cardiac flow rate based on a first signal and a second signal,
wherein the first signal is from a first optical sensor disposed on a first portion of a medical device having an elongated housing and being configured to be at least substantially entirely inserted within a vasculature of a patient, the first signal being indicative of a first red blood cell count, and wherein the second signal is from a second optical sensor disposed on a second portion of the medical device, the second signal being indicative of a second red blood cell count, the second portion of the medical device being longitudinally displaced from the first portion of the medical device.
